(19) United States
(12) Patent Application Publication
(10) Pub. No.: US 2026/0183305 A1
(43) Pub. Date: Jul. 2, 2026

(54) METHYLENE BLUE FOR USE IN THERAPY OF HEPATITIS B AND/OR HEPATITIS D INFECTION

(71) Applicant: Erich CERNY, Hergiswil (CH)

(72) Inventors: Andreas CERNY, Lugano (CH); Thomas CERNY, Goldiwil bei Thun (CH); Sophie CLÉMENT-LEBOUBE, Farges (FR); Francesco NEGRO, Perly (CH)

(21) Appl. No.: 19/133,077

(22) PCT Filed: Nov. 30, 2023
(86) PCT No.: PCT/EP2023/083665

§ 371 Date: May 27, 2025

(30) Foreign Application Priority Data
Nov. 30, 2022 (EP) 22210612.2

(51) Int. Cl. A61K 31/5415; A61K 9/00; A61K 9/08; A61K 9/20; A61K 31/513; A61K 31/522; A61K 31/675; A61K 31/7072; A61K 38/16; A61K 38/21; A61K 39/42; A61P 31/20

(52) U.S. Cl. CPC A61K 31/5415; A61K 9/0019; A61K 9/0043; A61K 9/0053; A61K 9/08; A61K 9/2009; A61K 9/2013; A61K 9/2054; A61K 9/2068; A61K 31/513; A61K 31/522; A61K 31/675; A61K 31/7072; A61K 38/16; A61K 38/212; A61K 39/42; A61P 31/20

(57) ABSTRACT

The present invention relates to the use of methylene blue in the treatment of treatment of hepatitis B (HBV) and/or hepatitis D (HDV) infection of a human patient as well as pharmaceutical compositions applicable in this treatment.

The present invention relates to the use of methylene blue in the treatment of hepatitis B (HBV) and/or hepatitis D (HDV) infection of a human patient as well as pharmaceutical compositions applicable in this treatment.

### BACKGROUND OF THE INVENTION:

Hepatitis B (HBV) and Hepatitis Delta (HDV) Virus, a satellite virus of HBV, are two of the five hepatitis viruses infecting humans providing worldwide a significant contribution to morbidity and mortality.

A highly effective prophylactic HBV vaccine exists, known antiviral drugs against HBV and HDV on the other hand need to be significantly improved.

Methylene Blue (MB) is the first synthetic molecule used in medicine and was initially synthesized by Heinrich Caro in 1876. The molecule has since found multiple applications in medicine, particularly in the field of antimalarial drugs, medication against methemoglobin intoxication or as a medication to treat Ifosfamide toxicity. The FDA and EMA approved drug MB is already widely used in clinical medicine for other applications, has few side effects and can be cheaply produced. In the field of virology, light activated MB was shown to clear in vitro blood supply from different infectious viral particles:

Wu W. et al., 2014, “Method of inactivating virus in circular blood and its applications in treating viral diseases”, U.S. Pat. No. 8,808,977, describes an in vitro procedure where MB is added to blood. The blood is then recirculated with the help of a pump under a light source until the virus is fully neutralized. A removing device is used to absorb the photosensitizer before infusing the blood.

Floyd R. et al., “Thiazine dyes used to inactivate HIV in biological fluids”, U.S. Pat. No. 5,827,644, describes an in vitro method using MB and light to treat biological fluids against human immunodeficiency virus.

Zepp Ch. et al., “Method for inactivating non-enveloped viruses using a viricide-potentiating agent with a photoactivatable virucide”, U.S. Pat. No. 5,663,043, describes a method for inactivating in blood non enveloped viruses with the help of a photoactivatable virucide, and the administration of a viricide-potentiating chemical agent such as for example a cationic lipo-polyamine.

Swartz M.R., “Method for inactivating viruses, bacteria, etc. in vitro and production of vaccines”, U.S. Pat. No. 4,402,318. A method of inactivating infectious agents in vitro is disclosed whereby MB is activated by concurrent application of an electric field and light.

Cerny E. H. et al., “ Methylenblau und Riboflavin zur prophylaktischen und therapeutischen antiviralen Therapie”, (MB and riboflavin for prophylactic and therapeutic antiviral therapy), Swiss patent CH717522A2. Cerny, E H et al. have shown that the prolonged incubation time corresponding to in vivo application as well as the body temperature of a mammal, deliver enough energy for virucidal treatment of MB against Corona or Influenza virus infection at physiological concentrations. The unusual long incubation times of in vivo application as compared to in vitro virus titration, i.e. days instead of minutes render this possible. A strong antiviral efficacy in the total dark (hermetically closed box) against Influenza N1H1 virus and SARS-COV-2 was demonstrated in vitro. This opens the possibility to use MB for in vivo treatment of Influenza A and Corona virus infections.

No prior art has shown antiviral efficacy against HBV nor HDV with MB. The fact that HDV infection is obligatory concomitant with an HBV infection poses a challenge for the development of antiviral medications, as efficient antiviral medications against two viruses simultaneously are required.

There are drugs, which inhibit efficiently HBV replication (nucleotide analogues such as Entecavir), but without therapeutic impact on HDV replication. A further complication is, that the two viruses are completely different: HBV is a Hepadnavirus with an outer lipid envelope and a nucleocapsid containing viral DNA and a DNA polymerase with reverse transcriptase activity. HDV, on the other hand, is a Ribozyviria virus, an extremely small negative-sense single-stranded RNA virus presenting a high polymorphism. The HDV virion is a spherical particle with a viral envelope containing host phospholipids, as well as three proteins taken from HBV. The envelope contains an inner ribonucleoprotein (RNP) particle and the genome is surrounded by hepatitis D antigen (HDAg). HDV has no druggable enzyme with the notable exception of its ribozyme activity.

There is, therefore, a need for a novel therapeutically approach allowing the treatment of HBV and/or HDV infections in human patients.

### SUMMARY OF THE INVENTION

The above-mentioned problem was, surprisingly, solved by the provision of a methylene blue based therapy of HBV and/or HDV infections as further defined herein below.

The present investigation of MB as an antiviral against HBV and HDV was initiated with the speculative line of reasoning, that the energy required for the transfer of oxygen from its triplet ground state to the singlet excited state is relatively low (96 KJ/mol) and in vivo treatment conditions, i.e. normal body temperature and prolonged duration of treatment, are sufficient to generate significant antiviral levels of singlet oxygen.

Furthermore, the in vitro testing of HDV infected cell cultures with MB was started with the expectation to observe an additional boost in antiviral activity against HDV because HDV has an exceptionally high percentage of guanine nucleobase in its genome (70%): a very important or the most important step for MB's antiviral activity is the oxidation of guanine to its 8-oxo-7,8-dihydroguanine form, a step due to the singlet oxygen generation by MB in the presence of oxygen.

During in vitro testing of MB with HBV envelope protein and HDV infected cell cultures, the inven-tors found to their surprise not only an antiviral activity against HDV but also a diminished secretion of HBV surface Antigen (HBVsAg) after treatment of the infected cells in cell culture with MB. The HDV RNA level as measured by a reverse Polymerase Chain Reaction (RT-PCR) decreased in a dose dependent manner, thereby demonstrating the efficacy of MB at physiological concentrations against HDV and even more surprisingly against HBV (FIGS. 3 and 4).

While an efficient prophylactic vaccine against HBV exists, the present invention teaches for the first time MB as a broad acting, largely nucleic acid sequence independent antiviral compound for a therapeutic application against HBV and/or HDV. This is an important advantage, as both viruses, but particularly HDV, have a significant potential to mutate.

MB shows absence of known serious side effects even at a very high dose and is an FDA and European Medical Agency approved drug for applications outside of virology. MB stains the urine, and after long term application sclera and in white skinned patients also the skin lightly blue, but the effect is reversible after discontinuation of treatment.

In view of the foregoing, MB will deliver a strong non-virus sequence specific broad antiviral activity against HBV and/or HDV as described in more detail below.

MB can, depending on concentration and reaction partner, reduce or oxidize a compound. MB is able to take electrons on its aromatic thiazine ring to be reduced to leukomethylene blue (MBH2) and transfer electrons to other compounds depending on the redox states and the concentration of MB. Singlet oxygen is in a quantum state where all electrons are spin paired and corresponds to the lowest exited state of the diatomic oxygen molecule. MB as a sensitizer in combination with oxygen and a source of energy results in production of singlet oxygen, a very reactive reaction partner which corrupts DNA, RNA or protein moieties by mechanisms such as guanine oxidation, thereby having a broad non sequence specific antiviral activity. Known antiviral molecular modifications include, but are not limited to a) 8-oxo-7,8-dihydroguanine (8-oxoGua) lesions, b) modified carbonyl moieties on proteins, c) single-strand breaks (ssb) in the RNA genome d) RNA-protein crosslinks. All quoted lesions are shown in the literature to correlate well with antiviral activity or activity against phages (Schneider, J. E., Jr., et al., Potential mechanisms of photodynamic inactivation of virus by methylene blue. I. RNA-protein crosslinks and other oxidative lesions in Q beta bacteriophage. Photochem Photobiol, 1998. 67(3): p. 350-7).

FIGS. 2, 3 and 4 as described in more detail below show a significant dose dependent antiviral activity in HBV and HDV infected cell cultures after treatment with Methylene Blue (MB) at physiological concentrations. MB induces the production of singlet oxygen, which in turn acts as an antiviral compound by corroding RNA and DNA nucleotides as well as protein moieties such as carbonyl groups.

### DESCRIPTION OF FIGURES

FIG. 1 shows the timeline of the experimental setting of FIGS. 3 and 4

FIG. 2: Cytotoxicity of MB on HepG2-NTCP (A) and HepNB2.7(B) evaluated by MTT. Cells were treated with decreasing concentrations of MB kept either in the dark or photo-activated under visible light.

FIG. 3: Inhibitory effect of MB on HDV replication on HepG2-NTCP cells. Cells infected with either 10 or 50 MOI were treated with MB (2.5 or 1.25 μg/ml) and HDV replication was assessed at 3 days (A) or 6 days (B) post infection by quantitative RT-PCR.

FIG. 4: Inhibitory effect of MB on HDV replication on HepNB2.7 cells. Cells infected with either 10 or 50 MOI were treated with MB (2.5 or 1.25 μg/ml) and HDV replication was assessed at 3 days (A) or 6 days (B) post infection by quantitative RC PCR. (C) The secretion of the HBV surface antigen (HBsAg) was measured in the supernatant of cells infected with 10, 50 or 100 MOI and treated with MB (2.5 or 1.25 μg/ml).

### DETAILED DESCRIPTION OF THE INVENTION

### (1) Materials, Terms, Definitions

Unless otherwise defined, all technical and scientific terms used herein have the same meaning as commonly understood by one of ordinary skill in the art to which this disclosure belongs. In case of any contradiction, the definition provided in this application shall prevail. When a trade name appears herein, it is intended to refer to its corresponding commercial product or its active ingredient. All patents, published patent applications, and publications cited herein are incorporated by reference.

### Methylene Blue:

“Methylene blue” (MB) refers to the FDA approved drug, (NDA 204630) Compound CID: 6099, MF: C16H18ClN3S, MW: 319.9 g/mol InChIKey: CXKWCBBOMKCUKX-UHFFFAOYSA-M, IUPAC Name: [7-(dimethylamino)phenothiazin-3-ylidene]-dimethylazanium, chloride. Absorption max: 668, 609 nm (PubChem data base) or its salts or derivates.

### “Methylene Blue Cation”:

3,7-Bis(dimethylamino)phenothiazin-5-ium; Methylthioninium; Compound CID: 4139, MF: C16H18N3S+ MW: 284.4 g/mol; InChIKey: RBTBFTRPCNLSDE-UHFFFAOYSA-N, IUPAC Name: [7-(dimethylamino)phenothia-zin-3-ylidene]-dimethylazanium.

MB may also exist in the form of its hydrate, which is also encompassed by the present invention. MB is a redox dye, which means that depending on concentration and reaction partner it can reduce or oxidize a compound. More precisely, it is able to take electrons on its aromatic thiazine ring to be reduced to leukomethylene blue (MBH2) and transfer electrons to other com-pounds depending on the redox states and the concentration of MB.

“Singlet oxygen” is oxygen in a quantum state, where all electrons are spin paired corresponding to the lowest exited state of the diatomic oxygen molecule. A sensitizer (such as methylene blue or riboflavin) in combination with oxygen and a source of energy results in the production of singlet oxygen, a very reactive reaction partner which corrupts DNA or RNA by mechanisms such as guanine oxidation thereby having a broad non sequence specific antiviral activity: Observed chemical lesions (induced by singlet oxygen) in a Q beta phage systems include: a) 8-oxo-7,8-dihydroguanine (8-oxoGua) lesions, b) modified carbonyl moieties on proteins, c) single-strand breaks (ssb) in the RNA genome d) RNA-protein crosslinks. (Schneider, J. E., Jr., et al., Potential mechanisms of photodynamic inactivation of virus by methylene blue. I. RNA-protein crosslinks and other oxidative lesions in Q beta bacteriophage. Photochem Photobiol, 1998. 67(3): p. 350-7).

Other pharmacological effects of MB found in the literature may also contribute to its antiviral efficacy or have a clinical beneficial effect:
Distributive (hypovolemic) shock: MB produces a vasoconstriction in distributive shock by inhibition of nitric oxide synthase and guanylate cyclase. This is a concomitant and unex-pected beneficial effect of MB, because end stage viral infections present often the clinical status of a distributive shock. (Porizka, M., et al., Methylene blue administration in patients with refractory distributive shock—a retrospective study. Sci Rep, 2020. 10(1): p. 1828, Jang, D. H., L. S. Nelson, and R. S. Hoffman, Methylene blue for distributive shock: a potential new use of an old antidote. J Med Toxicol, 2013. 9(3): p. 242-9)
Alzheimer's Disease: MB oxidizes cysteine sulfhydryl groups on tau-protein to keep tau monomeric. One preclinical treatment study in tauopathy mice reported anti-inflammatory neuroprotective effects mediated by the Nrf2/antioxidant response element (ARE); another reported insoluble tau reduction and a learning and memory benefit when given early.
Methemoglobinemia: MB acts by reacting within red blood cells to form leukomethylene blue, which is a reducing agent of oxidized hemoglobin converting the ferric ion (Fe+++) back to its oxygen-carrying ferrous state (Fe++).
As antimalarial agent: MB, a specific inhibitor of P. falciparum glutathione reductase has the potential to reverse CQ (chloroquine) resistance and it prevents the polymerization of haem into haemozoin similar to 4-amino-quinoline antimalarials.
Ifosfamide induced neurotoxicity: MB functions as an alternate electron acceptor. It acts to reverse the NADH inhibition caused by gluconeogenesis in the liver while blocking the transformation of chloroethylamine into chloroacetaldehyde. In addition, it inhibits various amine oxidase activities, which also prevents the formation of chloroacetaldehyde.

### Epidemic, Pandemic:

The rapid expansion of a disease to a large number of people in a given population within a short period of time is an epidemic. Encompassing multiple countries or more one calls it a pandemic. HBV and HDV can arise in clusters, an epidemic evolution is not typical.

### Hepatitis B Virus (HBV), Hepatitis D Virus (HDV):

A liver-specific bile acid transporter named the sodium taurocholate co-transporting polypeptide (NTCP) has been identified as the cellular receptor for HBV and its satellite, the hepatitis D virus (HDV).

HBV is a partially double stranded DNA virus, belonging to the genus Orthohepadnavirus, which is part of the Hepadnaviridae family. HDV comprises different species of negative-sense single-stranded, closed circular RNA viruses, classified together as the genus Deltavirus, within the realm Ribozyviria.

The classification of serotype and genotype of HBV and HDV should not play a major role in the therapeutic response to MB, because the mechanism of action is largely nucleic acid sequence independent.

Acute infection with HBV causes acute viral hepatitis, the chronic form of infection with HBV may lead to cirrhosis, hepatocellular carcinoma and increase the risk of associated pathologies. Concomitant infection of HBV with HDV increases the pathogenicity of the disease and decreases survival rate.

MOI (Multiplicity of Infection) describes the number of viral particles that can infect each cell in a tissue culture vessel.

The vaccine against HBV surface antigen (HBsAg), is an excellent prophylactic vaccine. PEG-ylated Interferon-alfa (PEG-IFN-α), Entecavir (ETV), and Tenofovir disoproxil fumarate (TDF) are the first-line agents currently used in the treatment of Hepatitis B disease.

Treatments for chronic hepatitis D include conventional or PEG-ylated interferon alpha therapy, whereby the benefit generally stops if the drug is discontinued. The antiviral Hepcludex (bulevirtide), which binds and inactivates NTCP has been recently approved to treat hepatitis D and B.

The human liver carcinoma cell line HepG2, expressing NTCP, provides a valuable tool for studying the basic biology of the viruses and developing treatments for HBV and HDV infection.

A cell line to study HBV infection is described in R. Yan, Y. Zhang et al. “Spinoculation Enhances HBV Infection in NTCP-Reconstituted Hepatocytes”. PLOS ONE 2015 Vol. 10 Issue 6. The publication reports the generation of HepG2-NTCP cell lines and the evaluation of spinoculation as a method to improve infection yields with HBV.

Youki Ueda shows that other cell lines than HepG2 such as A8.15.78.10 cells can be used to create cell lines with equal susceptibility to HBV infection as HepG2-NTCB cell lines (Ueda Y, Gu W et al., “A new hepatoma cell line exhibiting high susceptibility to hepatitis B virus infection”. Biochem Biophys Res Commun. 2019 Jul. 12;515(1):156-162).

Sun Y et al. give detailed instructions for the production of NTCP based HBV infections systems. Sun Y, Qi Y, Peng B, Li W. “NTCP-Reconstituted In Vitro HBV Infection System.” Methods Mol Biol. 2017;1540:1-14.

A NTCP stable HepG2 Cell Line generated by infecting HepG2 cells with the lentivirus NT-GFP (pLVX-IRES-ZsGreen) is commercially available from APM (catalogue number SKU T6190, Applied Biological Materials Inc., Richmond, Canada).

Another supplier of a NTCP Stable Hepg2 Cell Line is Biocat (Catalog Number T6190-GVO-ABM, BioCat GMBH, Heidelberg).

Cell lines and stem cells to study HDV infection are discussed in iPSCs for Studying Infectious Diseases, Volume 8 in Advances in Stem Cell Biology 2021, Pages 149-213 (Academic Press) which is hereby incorporated as a reference.

Ni et al. established an Huh-7-derived cell line (Huh-7-END) for continuous virion production by integrating the HDV antigenome and HBsAgs sequence (Ni, Y., Zhang et al. “Generation and characterization of a stable cell line persistently replicating and secreting the human hepatitis delta virus.” Sci Rep 9, 10021, 2019).

HepaRG cells become susceptible to HDV infection upon differentiation or ectopic NTCP expression (Ni et al., “Hepatitis B and D Viruses Exploit Sodium Taurocholate Co-transporting Polypeptide for Species-Specific Entry into Hepatocytes”, Gastroenterology, Volume 146, Issue 4,2014, Pages 1070-1083).

Both primary cultures of human hepatocytes (PHHs) and Primary cultures of Tupaia belangeri Hepatocytes (PTH), (a tree shrew hepatocyte model leading to the identification of NTCP as a receptor for HBV and HDV) are permissive to HDV infection (Verrier et al., “Cell Culture Models for the Investigation of Hepatitis B and D Virus Infection.” Viruses. 2016 Sep. 20;8(9):261, and Walter, E., et al., (1996), “Hepatitis B virus infection of tupaia hepatocytes in vitro and in vivo”. Hepatology, 24: 1-5.)

Self-Assembling Co-Cultured Primary Human Hepatocytes (SACC-PHHs) with stromal cells supporting a long-term HBV/HDV infection are described by Winer (Winer et al., “Analysis of Host Responses to Hepatitis B and Delta Viral Infections in a Micro-scalable Hepatic Co-culture System.” Hepatology. 2020 January;71(1):14-30).

Unzu et al. report on the infection and replication of PHHs-derived hepatic progenitors differentiated to hepatocyte-like cells with HDV (Unzu et al., “Pharmacological Induction of a Progenitor State for the Efficient Expansion of Primary Human Hepatocytes.” Hepatology. 2019 May; 69(5):2214-2231).

iPSC (induced Pluripotent Stem Cells)-derived Human pluripotent stem cell-derived Hepatocyte-Like Cells (HLCs) are sensitive to HDV infection (F. Lange et al. ,“ Hepatitis D virus infection of stem cell-derived hepatocytes triggers an IFN- and NFKB-based innate immune response unable to clear infection.” bioRxiv 2022.08.11.502443).

Hepatitis B surface antigen (HBsAg) is a protein on the surface of HBV, which is shed by infectious cells and detected in high levels in serum during acute or chronic hepatitis B virus infection. There are 3 forms of HBsAg and the largest one is needed to produce infected virus particles. HBsAg is produced in excess than needed for HBV virion production and the excess particles are secreted as HBsAg. HBsAg itself is used to make the prophylactic hepatitis B vaccine.

HBV infection can be detected by the detection of its nucleic acid, antigen or antibodies against one of its proteins. A comprehensive overview is provided by M. Krajden, G. McNabb et al., “The laboratory diagnosis of hepatitis B virus”. Can J Infect Dis Med Microbiol. 2005 March-April; 16(2): 65-72.

HDV infection is detected by anti-HDV immunoglobulin G (IgG) and immunoglobulin M (IgM), and confirmed by detection of HDV RNA in serum by RT-PCR (reverse transcription Polymerase Chain Reaction).

### Prophylactic Application and Therapeutic Application of an Antiviral Compound:

The excellent efficacy of the Hepatitis B vaccine makes it rather unlikely, that MB will be used as a prophylactic medication.

Prophylactic use of MB may be indicated for the rare cases where due to immune deficiency, the patient may not mount an efficient antibody or cytotoxic T cell answer. In the prophylactic case, the probate is not yet infected and in the therapeutic case the probate is already infected by the virus. The probate population for prophylactic use concerns a population known to be at elevated risk for viral infection such as the medical personal treating virus, intravenous drug users, persons living with infected people, persons with comorbidities (diabetes, leukemia, immunosuppression etc.), people of advanced age, immune compromised persons etc. Absence of side effects is for a prophylactic application particularly important, because this is in most cases a healthy population. MB colors urine blue and after a prolonged prophylaxis also colors the skin and sclera. This effect is fully reversible. In view of the cost and side effects of an antiviral compound such as MB, treatment is stopped in the prophylactic mode, when the viral exposition disappears and in the therapeutic mode, when diagnostic tests reveal that the virus is no more detectable.

### Antiviral Activity, Antiviral Efficacy:

This describes the pharmacological effect due to a compound, which diminishes the infectivity of the virus. The typical measure in vitro is the counting of PFU (Platelet Forming Unit) in a virus neutralization assay and LD₅₀ or a clinical substitute such as elevated temperature, in in vivo testing. Counting of PFU is also possible in in vivo experiments: after infection, an organ can be homogenized and the PFU/weight ratio can be determined. Furthermore, the man skilled in the art uses generally accepted statistical methods to express LD₅₀ in PFU and vice versa. In alter-native assays, quantitative measurement of viral nucleic acid or secreted viral antigen is often used for quantification of virus. For viruses no provoking a lysis of the infected cell, such as HBV and HDV, a quantitative RT-PCR specific for the nucleic acid sequence of the virus is used as a correlate for the quantity of virus present in the cells.

### Active Immunization (Vaccination), Passive Immunization:

Historically, in passive immunization convalescence serum (i.e. blood serum that is obtained from an individual who has recovered from an infectious disease and contains antibodies against the infectious agent of the disease) or serum produced by active immunization containing neutralizing antibodies is given to a patient to protect against infection. Convalescence serum can be replaced by neutralizing monoclonal antibodies or genetically engineered antibody like molecules directed against a neutralizing epitope of the virus.

MB can be applied in combination with active or passive immunization, any type of antiviral anti-body or any antiviral compound authorized for use against HBV or HDV. Passive immunization and or MB treatment of a newborn may also be envisioned in case of an infected mother. MB is known to be teratogenic and should not be used during pregnancy (Tiboni G M, Lamonaca D. Transplacental exposure to methylene blue initiates teratogenesis in the mouse: preliminary evidence for a mechanistic implication of cyclic GMP pathway disruption. Teratology. 2001; 64:213-220.)

### PEG-ylated Interferon and Small Molecule Antiviral Compounds for HBV and HDV:

MB is on theoretical grounds compatible with the use of interferon alpha-2a, PEG-ylated interferon alpha-2a. There are no in vivo or in vitro data available concerning the interaction of MB with other known antiviral compounds against HBV or HDV: lamivudine, adefovir, tenofovir disoproxil, tenofovir alafenamide, telbivudine, entecavir.

### Applied Dose of MB, Toxicity:

The invention describes a compound containing MB for the prophylaxis and/or treatment of HBV and/or HDV virus infections of humans acting by its antiviral efficacy after application by the oral, intravenous, subcutaneous, intra muscular, intra nasal, rectal or through the nose or mouth, whereby the daily dose is not less than 0.1 milligram per application and not higher than 20 milligram per kg bodyweight of the patient and 24 hours.

MB may precipitate serotonin syndrome and should not be given together with serotonin reuptake inhibitors. It can cause hemolytic anemia in patients with glucose-6-phosphate dehydrogenase (G6PD) enzymatic deficiency at high dose. Gastrointestinal symptoms may appear at higher oral dose due to the bitter taste which can be masked by galenic formulation. During World War I, soldiers received more than 400 mg of MB per day over several weeks for malaria prophylaxis without major side effects (Marshall D G. The “toxicity” of methylene-blue. Lancet. 1920;195(5051):1334). Brazilian children were reported to tolerate 20-50 mg/kg per day of MB very well for long periods of time (Ferreira M C. Sur l'emploi du bleu de méthylène dans la malaria infantile. Ther Medico-Chirugicale. 1893;124:488-525). In general, MB given orally seems to be largely well tolerated, MB given intravenously must be applied with caution. For sheep, the LD50 of MB was found to be 42 mg/kg when applied intravenously (Burrows G E. Methylene blue: effects and disposition in sheep. J Vet Pharmacol Ther. 1984;7(3):225-231).

### Virology Methods:

The man skilled in the art is familiar with the widely used virology methods described herein. The book “Diagnostic Virology Protocols” , 2011, edited by John R. Stephenson and Alan Warnes, Springer Verlag, is one of many comprehensive manuals for the methods to study, manipulate, and detect viruses. This text book completes the concise description of the methods and procedures given here and its content is herewith incorporated by reference.

### (2) Embodiments of the Invention

### (2.1) Particular Embodiments

The present invention relates to the following aspects and embodiments thereof:

According to a first aspect, the present invention relates to a methylene blue (MB) compound or an active ingredient containing MB for use in the therapeutic or prophylactic treatment, in particular in the therapeutic treatment, of hepatitis B (HBV) and/or hepatitis D (HDV) infection of a human patient.

According to a particular embodiment of thereof, a HDV infection is treated.

According to another particular embodiment thereof, a HBV infection is treated.

According to another particular embodiment thereof, a combined, i.e. simultaneous or sequential, infection of HBV and HDV is treated.

In still another particular embodiment of said first aspect, the virus is a genotype of HBV, of HDV, or of HBV and HDV.

According to still another particular embodiment, the treatment is a therapeutic treatment of such infection.

According to still another particular embodiment, the treatment is a prophylactic treatment of such infection.

According to still another particular embodiment said compound or an active ingredient containing MB essentially consists of MB as defined above, in particular any solid form of MB, and/or hydrate thereof.

In particular, any anionic salt form of MB is applied. Non-limiting examples of suitable and unique salts are selected from acetate, acistrate, besylate, bromide, chloride, citrate, fumarate, glucouronate, hydrobromide, hydrochloride, hydroiodide, iodide, lactate, maleate, mesylate, nitrate, pamoate, phosphate, succinate, sulfate, tartrate, tosylate, and xinofoate. More particular, the aninic salt is the chloride salt of MB.

According to still another particular embodiment, said the compound or active ingredient containing MB is acting by its antiviral efficacy.

According to still another particular embodiment the treatment is for the purpose of

According to another particular embodiment, said compound or an active ingredient containing MB is applied to the patient, for prophylactic treatment by the oral route, intranasal application, through intravenous, subcutaneous or intra muscular injection, by the rectal or nebulizer route or any combination thereof, in particular by the oral and intravenous route.

According to another particular embodiment, said compound or an active ingredient containing MB is applied to the infected patient for therapeutic treatment by the oral route, intranasal application, through intravenous, subcutaneous or intra muscular injection, by the rectal or nebulizer route or any combination thereof, in particular by the oral and intravenous route.

In a more particular embodiment of said first aspect, the oral route of administration is applied for MB.

In still another more particular embodiment, the oral route of administration is performed by applying a solid formulation which is encapsulated, in particular, in order to mask the bitter taste of MB.

In still another more particular embodiment, said orally administered formulation allows for a slow release of MB.

According to another particular embodiment, the daily dose of said compound or active ingredient containing MB administered for anyone of the above medical purposes, is in the range of 0.1 mg to 20 mg per kg bodyweight of the human patient.

According to further particular embodiments, said compound or an active ingredient containing MB is administered
a) orally, particularly in a daily dose of 0.1 to 20, more particularly 0.5 to 7.5, even more particularly 1 to 5, most particularly 2 to 4 mg/kg bodyweight per day of the patient; or
b) by injection particularly in a daily dose of 0.1 to 20, more particularly 0.5 to 7.5, even more particularly 1 to 5, most particularly 2 to 4 mg/kg bodyweight per day of the patient; or
c) rectally, particularly in a daily dose of 0.1 to 20, more particularly 0.5 to 7.5, even more particularly 1 to 5, most particularly 2 to 4 mg/kg bodyweight per day of the patient.

According to further particular embodiments, said compound or an active ingredient containing MB is administered
d) via nebulizer, particularly in a daily dose of 0.1 to 20 or 0.1 to 10, more particularly 0.5 to 7.5, even more particularly 1 to 5, most particularly 2 to 4 mg/kg bodyweight of the patient; or
e) intranasally, particularly in a daily dose of 0.1 to 20 or 0.1 to 10, more particularly 0.5 to 7.5, even more particularly 1 to 5, most particularly 2 to 4 mg/kg bodyweight of the patient.

Most particularly, the dose for an antiviral treatment via the oral route is 3 mg/kg in adults per 24 hours. The maximum dose should not exceed 10 mg/kg per day if given over an extended period of time, as for example more that 5 to 10 days.

In still another particular embodiment of said first aspect said compound or an active ingredient containing MB administered by injection, in particular intravenously, more particularly by infusion, in a daily dose of 0.1 to 10, more particularly 0.5 to 7.5, even more particularly 1 to 5, most particularly 2 to 4 mg/kg bodyweight of the patient.

Most particularly, the dose for antiviral treatment via injection, in particular infusion, is 3 mg/kg in adults per 24 hours. If given as a bolus it should be applied over a period of at least 5 minutes. The maximum dose should not exceed 10 mg/kg per day if given over an extended period of time, as for example more that 5 to 10 days.

In still another particular embodiment of said first aspect said compound or an active ingredient containing MB is administered by a combination of at least two routes of administration selected from the group of oral, intravenous, subcutaneous, intra muscular, intranasal or nebulizer route, particularly by a combined daily dose of 0.1 to 20 or 0.1 to 10, more particularly 0.5 to 7.5, even more particularly 1 to 5, most particularly 2 to 4 mg/kg bodyweight per day of the patient.

In still another particular embodiment of said first aspect said compound or an active ingredient containing MB is administered via anyone of the above routes of administration, when the viral infection is caused by a HBV; or when the viral infection is caused by a simultaneous or sequential infection through a HBV and a HDV.

In still another particular embodiment of said first aspect said compound or an active ingredient containing MB is administered via anyone of the above routes of administration in viral infection induced distributive shock acting on vasoconstriction of small vessels due to its effect on nitric oxide.

In still another particular embodiment of said first aspect said compound or an active ingredient containing MB is administered via anyone of the above routes of administration concomitantly (i.e. simultaneously or sequentially in any order) with one or more further therapeutic agents, in particular selected from interferon alpha-2a, PEG-ylated interferon alpha-2a, interferon alpha-2b, PEG-ylated interferon alpha-2b, 2b, or any small molecule effective for antiviral medications against HBV and/or HDV, such as lamivudine, adefovir, tenofovir disoproxil, tenofovir alafenamide, telbivudine, bulevirtide and entecavir.

In still another particular embodiment of said first aspect said compound or an active ingredient containing MB is administered via anyone of the above routes of administration for a therapeutic use, wherein the treatment is performed over a period of at least one day, particularly for at least one month, more particularly for 3 to 24 months, even more particularly for 6 to 18 months, most particularly 12 months, or until a negative virological test result for the presence of HBV and/or HDV in the patient is obtained.

In still another particular embodiment of said first aspect said compound or an active ingredient containing MB is administered via anyone of the above routes of administration, wherein the treatment is performed in the absence of an external, i.e. extra-corporal, high-energy light source activating methylene blue.

In still another particular embodiment of said first aspect said compound or an active ingredient containing MB is administered via anyone of the above routes of administration in the form of a liquid pharmaceutical composition.

Particularly, said pharmaceutical composition is in solid or liquid form, comprising in a pharmaceutically acceptable carrier or diluent a viricidally effective amount of said compound or an active ingredient containing MB.

More particularly, said pharmaceutical composition comprises said compound or an active ingredient containing MB in a liquid pharmaceutically acceptable carrier in a proportion in the range of 0.1 to 2 wt.-%, particularly 0.5 to 1.5 wt.-%, and more particularly 0.8 to 1.2 wt.-%, and especially about 1 wt.-%, based on the total weight of the composition.

More particularly, said pharmaceutical composition comprises said compound or an active ingredient containing MB in a solid pharmaceutically acceptable carrier in a proportion in the range of 0.1 to 2 wt.-%, particularly 0.5 to 1.5 wt.-%, and more particularly 0.8 to 1.2 wt.-%, and especially about 1 wt.-%, based on the total weight of the composition.

According to a second aspect, the present invention relates to a solid or liquid pharmaceutical composition as defined above.

According to a third aspect, the present invention relates to a method for the therapeutic treatment of hepatitis B (HBV) and/or hepatitis D (HDV) infection of a human patient, which method comprises administering to the patient a viricidally effective amount of a methylene blue compound as further defined above for the first aspect of the invention

More particularly, the use, compositions or method of anyone of the preceding aspects or embodiments, applies MB in essentially pure form, in particular a pharmaceutically acceptable salt or hydrate thereof.

More particularly, during the use or method of anyone of the preceding aspects or embodiments, said MB treatment is applied in combination with active or passive immunization, with any type of antiviral antibody and/or with any type of antiviral compound effective against HBV and/or HDV, such as lamivudine, adefovir, tenofovir disoproxil, tenofovir alafenamide, telbivudine, bulevirtide and entecavir.

### (2.2) Further Embodiments

One or more compounds or “active agents” disclosed herein can be administered to a patient by themselves or in pharmaceutical compositions where they are mixed with biologically suitable carriers or excipient(s) at doses effective to prevent, treat, attenuate or ameliorate a disease or condition as described herein. Mixtures of these compounds can also be administered to the patient as a simple mixture or in suitable formulated pharmaceutical compositions.

“Patient” as used herein means human or non-human, in particular human, animals.

An “active agent” or “compound” in the context of the present invention means any compound, element, or mixture that when administered to a patient alone or in combination with another agent confers, directly or indirectly, a physiological effect on the patient. When the active agent is a compound, salts, solvates (including hydrates) of the free compound or salt, crystalline and non-crystalline forms, as well as various polymorphs of the compound are included. Compounds may contain one or more asymmetric elements such as stereogenic centers, stereogenic axes and the like, e.g. asymmetric carbon atoms, so that the compounds can exist in different stereo isomeric forms. These compounds can be, for example, racemates or optically active forms. All stereoisomers, diastereomers, Z- and E-forms, in purified and mixture forms are included. Accordingly, when a compound is recited by specific name or a class of compounds is recited, all these forms are intended to be included.

A “dosage form” is any unit of administration (“unit dose”) of one or more active agents as described herein.

The term “treating” or “treatment” refers to: (i) preventing a disease, disorder or condition from occurring in a patient which may be exposed or predisposed to the disease, disorder and/or condition but has not yet been diagnosed as having it; (ii) inhibiting the disease, disorder or condition, i.e., arresting its development; and (iii) relieving the disease, disorder or condition, i.e., causing regression of the disease, disorder and/or condition.

The compounds or active ingredients of this invention are generally given as pharmaceutical compositions comprised of a prophylactically or therapeutically effective amount of at least one such compound or its pharmaceutically acceptable salt and a pharmaceutically acceptable carrier and may contain conventional excipients.

The phrase “pharmaceutically acceptable” is employed herein to refer to those compounds, materials, compositions, and/or dosage forms which are, within the scope of sound medical judgment, suitable for use in contact with the tissues of patients without excessive toxicity, irritation, allergic response, or other problem or complication commensurate with a reasonable risk/benefit ratio.

The invention includes all “pharmaceutically acceptable salt forms” of the compounds. Pharmaceutically acceptable salts are those in which the counter ions do not contribute significantly to the physiological activity or toxicity of the compounds and as such function as pharmacological equivalents. These salts can be made according to common organic techniques employing commercially available reagents. Some anionic salt forms include acetate, acistrate, besylate, bromide, chloride, citrate, fumarate, glucouronate, hydrobromide, hydrochloride, hydroiodide, iodide, lactate, maleate, mesylate, nitrate, pamoate, phosphate, succinate, sulfate, tartrate, tosylate, and xinofoate. Some cationic salt forms include ammonium, aluminum, benzathine, bismuth, calcium, choline, diethylamine, diethanolamine, lithium, magnesium, meglumine, 4-phenylcyclohexylamine, piperazine, potassium, sodium, tromethamine, and zinc.

A “therapeutically effective amount” means an amount effective, when administered to a human or non-human patient, to provide any therapeutic benefit. More particularly, a “therapeutically effective amount” is an amount of a compound disclosed herein or a combination of two or more such compounds, which inhibits, totally or partially, the progression of the condition or alleviates, at least partially, one or more symptoms of the condition.

A therapeutic benefit may be an amelioration of symptoms of a diseased patient, e.g., an amount effective to decrease the symptoms of the patient or laboratory indicators of liver function. The final goal is the eradication of HBV and/or HDV.

“Frequency” of dosage may vary depending on the compound used and the particular type of infection treated. A dosage regimen of once per day or an even longer period is possible. Dosage regimens in which the active agent is administered for several times daily, as for example 2 to 10 times, like 2, 3, 4, 5, 6, 7, 8, 9 or 10 times may occasionally be more helpful.

It will be understood, however, that the specific dose level and frequency for any particular patient will depend upon a variety of factors including the activity of the specific compound employed, the age, body weight, general health, sex, diet, time of administration, route of administration, rate of excretion, drug combination and the severity of the particular disease in the patient undergoing therapy. Patients may generally be monitored for therapeutic effectiveness using assays suitable for the condition being treated, which will be familiar to those of ordinary skill in the art.

Solid compositions are normally formulated in dosage units and compositions providing from about 0.1 to 2000 mg of the active ingredient per dose are of interest. Some examples of dosages are 1 mg, 10 mg, 25 mg, 50 mg, 100 mg, 250 mg, 500 mg, and 1000 mg.

Liquid compositions are usually in dosage unit ranges. Generally, the liquid composition will be in a unit dosage range of 1-100 mg/ml. Some examples of dosages are 1 mg/ml, 10 mg/mL, 25 mg/mL, 50 mg/mL, and 100 mg/ml.

The invention also encompasses methods where the compound is given in combination therapy. That is, the compound can be used in conjunction with, but separately from, other agents useful in treating infection. In these combination methods, the compound will generally be given in a daily dose as specified above in conjunction with other agents. The other agents generally will be given in the amounts used therapeutically. The specific dosing regimen, however, will be deter-mined by a physician using sound medical judgment.

For any compound or combination thereof used according to the present invention, the therapeutically effective dose can be estimated initially from cellular assays or animal models. For example, a dose can be formulated in cellular and animal models to achieve a circulating concentration range that includes the IC₅₀ as determined in cellular assays (i.e., the concentration of the test compound which achieves a half-maximal inhibition of a given activity). In some cases, it is appropriate to determine the IC₅₀ in the presence of 3 to 5% serum albumin since such a determination approximates the binding effects of plasma protein on the compound. Such information can be used to more accurately determine useful doses in humans.

Toxicity and therapeutic efficacy of such compounds or combination thereof can be determined by standard pharmaceutical procedures in cell cultures or experimental animals, e.g., for determining the Maximum Tolerated Dose (MTD) and the ED₅₀ (Effective Dose for 50% maximal response). The dose ratio between toxic and therapeutic effects is the therapeutic index and it can be expressed as the ratio between MTD and ED₅₀. Compounds or combinations thereof, which exhibit high therapeutic indices are preferred. The data obtained from these cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. The exact formulation, route of administration and dosage can be chosen by the individual physician in view of the patient's condition (see e.g. Fingl et al., 1975, in “The Pharmacological Basis of Therapeutics”, Ch. 1 p. 1).

Dosage amount and interval may be adjusted individually to provide plasma levels of the active moiety, which are sufficient to maintain the desired effects, or Minimal Effective Concentration (MEC). The MEC will vary for each compound but can be estimated from in vitro data; e.g. the concentration necessary to achieve 50-90% inhibition of protein kinase using the assays described herein. Dosages necessary to achieve the MEC will depend on individual characteristics and route of administration. However, HPLC assays or bioassays can be used to determine plasma concentrations.

Dosage intervals can also be determined using the MEC value. Compounds should be administered using a regimen, which maintains plasma levels above the MEC for 10-90% of the time, preferably between 30-90% and most preferably between 50-90% until the desired amelioration of symptoms is achieved.

The amount of composition administered will, of course, be dependent on the subject being treated, on the subject's weight, the severity of the affliction, the manner of administration and the judgment of the prescribing physician.

The term “pharmaceutical composition” means a composition comprising at least one pharmaceutically active compound as described herein in combination with at least one additional pharmaceutical carrier, i.e., adjuvant, excipient or vehicle, such as diluents, preserving agents, filers, stabilizers, extenders, binders, humidifiers, flow regulating agents, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms. Ingredients listed in Remington's Pharmaceutical Sciences, 18th ed., Mack Publishing Company, Easton, PA (1999) for example, may be used.

A pharmaceutical composition as used herein may be presented in the form of a “dosage form” or “unit dose” and may comprise one or more active agents. Thus, a pharmaceutical composition as used herein could, for example, provide two active agents admixed together in a unit dose or provide two active agents combined in a dosage form wherein the active agents are physically separated and/or have different release rates.

A “combined pharmaceutical product” as used herein is a combination of two more doses of two or more different active agents combined in separate dosage forms, which are not admixed.

Pharmaceutical compositions include any suitable “formulation” including, for example, capsules, tablets, coated tablets, injections and liquids and may be administered through any suitable route.

Suitable routes of administration may, for example, include oral, rectal, transmucosal or intestinal administration; parenteral delivery, including intramuscular, subcutaneous, intramedullary injections, as well as intrathecal, intravenous or intraperitoneal, applications.

Furthermore, one may administer the drug in a targeted drug delivery system, for example, in a liposome coated with endothelial cell-specific antibody or peptide.

The pharmaceutical compositions of the present invention may be manufactured in a manner that is itself known, e.g., by means of conventional mixing, dissolving, granulating, dragee making, levigating, emulsifying, encapsulating, entrapping or lyophilizing processes. Proper formulation is dependent upon the route of administration chosen.

For injection, and for application in liquid form, the agents of the invention may be formulated in aqueous solutions, preferably in physiologically compatible buffers such as Hanks'solution, Ringer's solution, or physiological saline buffer.

For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds disclosed herein to be formulated as tablets, pills, dragees, capsules, liquids, gels, syrups, slurries, suspensions and the like, for oral ingestion by a patient to be treated. Pharmaceutical preparations for oral use can be obtained by combining the active compound with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. Coatings are particularly important for MB in view of the bitter taste, which may cause gastrointestinal symptoms and lower patient compliance with treatment schedule. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such admin-istration.

The compounds can be formulated for parenteral administration by injection, e.g. bolus injection or continuous infusion. Formulations for injection may be presented in unit dosage form, e.g. in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances, which increase the viscosity of the suspension, such as sodium carboxymethyl cellulose, sorbitol, or dextran. Optionally, the suspension may also contain suitable stabilizers or agents, which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides. Rectal application is attractive, because the problems due to the bitter taste of MB are circumvented.

In addition to the formulations described previously, the compounds may also be formulated as a depot preparation. Such long-acting formulations may be administered by injection or implantation (for example subcutaneously or intramuscularly). Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

An example of a pharmaceutical carrier for the hydrophobic compounds disclosed herein is a cosolvent system comprising benzyl alcohol, a nonpolar surfactant, a water-miscible organic polymer, and an aqueous phase. The cosolvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethysulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained release capsules may, depending on their chemical nature, release the compounds for a few hours up to over several days.

The pharmaceutical compositions may also comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Compositions comprising a compound disclosed herein formulated in a compatible pharmaceutical carrier may also be prepared, placed in an appropriate container, and labelled for treatment of an indicated condition. Commercial products approved and marketed in Switzerland suitable (but not destinated or evaluated) for HBV and HDV treatment include “Proveblue™”, containing 50 mg MB diluted in 10 ml solution for injection approved for treatment of methemoglobinemia toxicity and “Lumeblue™”, Cosmo Pharmaceuticals, 25 mg coated tablets for diagnostic bowel lining coloring during coloscopy.

In some formulations it may be beneficial to use the compounds disclosed herein in the form of particles of very small size, for example as obtained by fluid energy milling.

The use of compounds disclosed herein in the manufacture of pharmaceutical compositions is illustrated by the following description. In this description the term “active compound” denotes any compound of the invention but particularly any compound which is the final product of one of the following nonlimiting examples.

### a) Capsules

In the preparation of capsules, 100 parts by weight of active compound and 150 parts by weight of lactose can be de-aggregated and blended. The mixture can be filled into hard gelatin capsules, each capsule containing a unit dose or part of a unit dose of active compound.

### B) Tablets

Tablets can be prepared, for example, from the following ingredients.

Parts by weight: Active compound 100, Lactose 150, Maize starch 22, Polyvinylpyrrolidone 10, Magnesium stearate 3 parts. The active compound, the lactose and some of the starch can be de-aggregated, blended and the resulting mixture can be granulated with a solution of the polyvinylpyrrolidone in ethanol. The dry granulate can be blended with the magnesium stearate and the rest of the starch. The mixture is then compressed in a tableting machine to give tablets each containing a unit dose or a part of a unit dose of active compound.

### c) Enteric Coated Tablets

Tablets can be prepared by the method described in (b) above. The tablets can be enteric coated in a conventional manner using a solution of 20% cellulose acetate phthalate and 3% diethyl phthalate in ethanol: dichloromethane (1:1).

### d) Suppositories

In the preparation of suppositories, for example, 100 parts by weight of active compound can be incorporated in 1300 parts by weight of triglyceride suppository base and the mixture formed into suppositories each containing a therapeutically effective amount of active ingredient.

The present invention will now be described in more detail by reference to the following nonlimiting, illustrative examples.

### Materials

### a) Chemicals

The compound methylene blue (Methylthonium chloride solution) was purchased from ProVepharm, Marseille, France.

All other chemicals as applied herein were of analytical grade and are commercially available products.

Cell culture media and supplements as used herein are commercially available products.

### b) Viral Particles

HDV particles were produced and tittered as previously described (Alfaiate D, Lucifora J et al; “HDV RNA replication is associated with HBV repression and interferon-stimulated genes induction in super-infected hepatocytes.” Antiviral Res 2016; 136:19-31.).

### c) Cell Lines and Cell Culture

HepG2-NTCP is a hepatoma derived cell line expressing NCTP, the cellular receptor of HBV and HDV. When infected with HDV, there is a replication of HDV but no production of new viruses (Ni, Y. et al. Hepatitis B and D viruses exploit sodium taurocholate co-transporting polypeptide for species-specific entry into hepatocytes. Gastroenterology 146, 1070-1083 (2014)).

HepNB2.7 is a hepatoma derived cell line with constitutive expression of the HBV envelope proteins (HBsAg) but no expression of other proteins required for production of HBV particles. When infected with HDV, there is a replication of HDV and production and new HDV particles. Production of new HDV particles requires the expression of HBsAg by the cell.

The cell lines have been developed specifically for the detection and evaluation of antiviral compounds against HBV and HDV (Lempp F A et al., “Recapitulation of HDV infection in a fully permissive hepatoma cell line allows efficient drug evaluation”. Nat Commun 2019;10:2265).

HepG2-NTCP cells and HepNB2.7 cells (each provided by Prof. Stephan Urban, University Hospital Heidelberg, Germany) were cultured in Dulbecco's Modified Eagle's medium (DMEM) supplemented with 10% fetal bovine serum (FBS), 1% L-glutamine, 1% penicillin-streptomycin (all from Gibco, Thermo Fisher Scientific) and 5 μg/ml of puromycin (ant-pr-1, InvivoGen).

Examples of alternative cellular test systems suitable for evaluating the efficacy of MB to treat or prevent HBV and HDV infections are provided above in the general part of the description.

### Methods

### a) Cell Infection and Cell Analysis

HepG2-NTCP or HepNB2.7 cells were infected with different titers of HDV (10, 50 MOI) and treated with 2.5 or 1.25 μg/ml of MB for 5 hours.

Cells were analyzed at day 3 or day 6 post-infection according to the MTT assay protocol (see below).

### b) MTT Cell Viability Assay

Cultures were maintained at 37° C. in a humidified atmosphere of 5% CO₂. Cell viability was assessed using the MTT assay (Sigma-Aldrich, In vitro Toxicology Assay Kit, TOX1-1KT) hepG2-NTCP and HepNB 2.7 cells plated at 10x 103 cells/well in 96 well plates 16 h prior to treatment for 6 days with different doses of MB. Cells were incubated at room temperature in the dark or under visible light (to photo-activate methylene blue) for different periods of time (as indicated for the respective experiments below; 1 hour/3 days/6 days). The assay uses the MTT reagent (3-(4,5-dimethylthiazol-2-yl)-2,5-diphenyltetrazolium bromide), to determine mammalian cell viability.

### c) Viral Infection and MB HDV Inhibition Assay

HepG2-NTCP and HepNB2.7 cells (plated at 10×10³ cells/well) were infected with 10 or 50 MOI of HDV using PEG (polyethylene glycol) 4% overnight as previously described (Alfaiate D, Lucifora J et al.; cf. above). MB (2.5 or 1.25 μg/ml) was added 16 hrs post HDV infection for 5 h and cells were incubated at room temperature in the dark. Three and six days post infection, RNA was extracted and HDV RNA level was monitored by RT-qPCR in order to determine HDV replication as described below. For HepNB2.7, the level of HBsAg in the supernatant were measured as described below.

### d) HBsAg Measurement

For HepNB2.7, the level of HBsAg in the supernatant were measured at both time points by ELISA (Enzyme-Linked Immosorbent Assay, ABBOTT PRISM® HBsAg) according to the protocol of the manufacturer.

### e) HDV Replication Quantification:

Total intracellular RNA was extracted using the Nucleospin RNA Kit (Macherey-Nagel AG). cDNA was synthesized from 100 ng total RNA using Superscript II and random hexamer primers (Roche Diagnosis). Quantification of HDV was performed by qRT-PCR as described (Alfaiate D, Lucifora J et al), using the primers described by Scholtes & colleagues (Scholtes C, Icard V, Amiri M et al; “Standardized one-step real-time reverse transcription-PCR assay for universal detection and quantification of hepatitis delta virus from clinical samples in the presence of a heterologous internal-control RNA.” J Clin Microbiol 2012;50:2126-2128.) and EEF1A1 as housekeeping gene.

By applying the above-described materials and methods, the following experiments were performed:

### Example 1: Investigating Cytotoxicity of MB on Hepatoma Cell Lines HepG2-NTCP and HepNB2.7

Cytotoxicity of MB on HepG2-NTCP and HepNB2.7 were evaluated by MTT. Cells were treated with decreasing concentrations of MB either kept in the dark or photoactivated under visible light.

The MTT Cell Viability Assay uses the MTT reagent (3-(4,5-dimethylthiazol-2-yl)-2,5-diphenyltetrazolium bromide), to determine mammalian cell viability. This assay's use in infected and non infected cells is important to prove, that the cytotoxic effect measured is due to the antiviral activity of the drug and not due to the direct cytotoxic effect on the cultured cells due to the drug under evaluation. The redox potential in active mammalian cells reduces MTT to a strongly pigmented formazan product. After solubilization, the absorbance of the formazan product can be measured with a microplate absorbance reader.

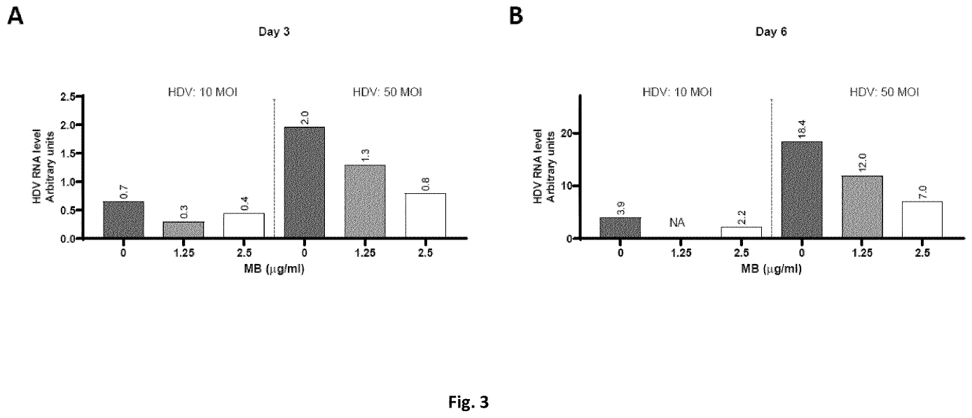

FIG. 2 shows the cytotoxicity of MB on HepG2-NTCP (A) and HepNB2.7(B) evaluated by MTT. Cells were treated with decreasing concentrations of MB either kept in the dark or photoactivated under visible light. The figure shows the percentage of viability of HepG2-NTCP (A) and HepNB2.7(B) cells 3 days after incubation with different concentrations of MB. The cytotoxic effect of MB is not significantly enhanced in the presence of light as compared to dark, but the long-term effect (3 days) shows significant toxicity.

### Example 2: Investigating the Inhibitory Effect of MB on HBV/HDV Infection

HepG2-NTCP or HepNB2.7 cells were infected with different titers of HDV (10, 50, 100 MOI) and treated with 2.5 or 1.25 μg/ml of MB for 5 hours. Cells were analyzed at day 3 or day 6 post-infection and HDV replication was assessed by RT-PCR. For HepNB2.7, the level of HBsAg in the supernatant were measured at both time points.

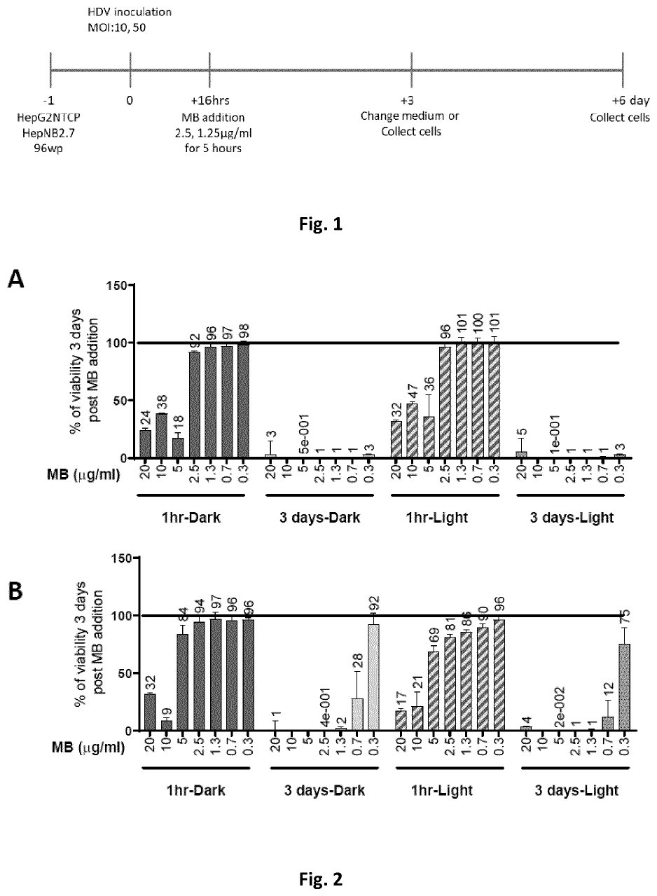

FIG. 1 shows the general timeline of the experimental setting of the these experiments.

The following experiments were performed with infected HepG2-NTCP or HepNB2.7 cells:

### a) Inhibitory Effect of MB on HDV Replication on HepG2-NTCP Cells.

The inhibitory effect of MB on HDV replication on HepG2-NTCP cells was investigated. Cells infected with either 10 or 50 MOI of virus were treated with MB (2.5 or 1.25 μg/ml) and HDV replication was assessed at 3 days (A) or 6 days (B) post infection.

FIG. 3 shows the significant antiviral effect of MB against HDV infected HepG2-NTCP cells at physiological drug concentrations. The discovery of the NTCP receptor as common entry for HBV and HDV infection of hepatocytes let to the cloning of NTCP-overexpressing HepG2 cells susceptible to HBV and HDV infection. This provided an HDV-susceptible cancer-derived cell line suitable for high-throughput studies. Cells were infected with either 10 or 50 MOI and were treated with MB (2.5 or 1.25 μg/mL) for 3 to 6 days. The HDV RNA concentration as measured shows a clear dose response relationship at 50 MOI at 3 and 6 days post infection. The HDV RNA concentration at 10 MOI are too low to distinguish the antiviral impact of MB.

### b) Inhibitory Effect of MB on HDV Replication on HepNB2.7 Cells.

The inhibitory effect of MB on HDV replication on HepNB2.7 cells was investigated. In a first experiment cells infected with either 10 or 50 MOI were treated with MB (2.5 or 1.25 μg/ml) and HDV replication was assessed at 3 days, or 6 days, post infection.

In a second experiment the secretion of the HBV surface antigen (HBsAg) was measured in the supernatant of cells infected with 10, 50 or 100 MOI and treated with MB (2.5 or 1.25 μg/ml).

The dose related effect of MB on viral reproduction under physiological conditions on HDV at 50 MOI and day 6 post infection is demonstrated on HepNB2.7 cells.

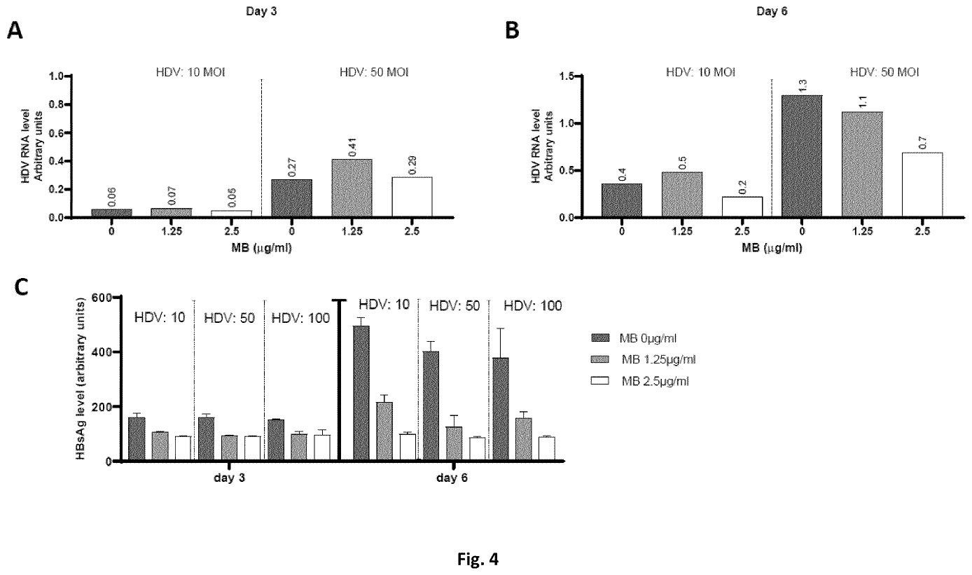

The results are shown in FIG. 4. The weak signal at day 3 and 6 at 10 MOI is due to the low infectious challenge (panels A and B). Panel C shows the clear-cut dose/response relationship for the antiviral activity of MB on viral replication,-using HBsAg as a proxy for the HBV virus, bus also the HBV dependent HDV virus-under all conditions tested. The antiviral effect of MB accumulates over time as shown by the results on day 3 and 6. Extrapolating the present data it seems reasonable to expect under conditions of long-term treatment (weeks instead of days) a complete disappearance of viral markers (with the exception of the constitutive part of HBV) from the cell lines (no data shown).

### Example 3: Methylene Blue, Galenic Formula for Different Routes of Application

In the following section, non-limiting examples of suitable MB formulations applicable in the treatment of HDV/HBV infections are described.

### a) Formula for Injection (Intravenous, Subcutaneous or Intra Muscular Application):

Methylthioninium chloride (MB, chloride salt) is diluted in distilled water as a solution for intravenous injection at a concentration of 5 mg/ml. The ionic strength of pure water is adjusted with KCl, because chloride ions reduce the solubility. The pH value is adjusted to pH=4.5 because long term stability is compromised at higher pH. The solution is stored in dark glass ampoules of 5, 10 and 20 mL volume. Dark glass, because of MB's light sensitivity. The solution is compatible with 5% glucose or 5% dextrose solution, but is not compatible with 0.9% saline solution due to the risk of precipitation.

The recommended dose for antiviral treatment is 1 to 10 mg/kg in adults per 24 hours. If given as a bolus it should be applied over a period of at least 5 minutes. The maximum dose should not exceed 5 mg/kg in case of application as a bolus. Caution is advised in the case of impaired renal function. Sensitivity to thiazine dyes and G-6-PD deficiency are further contraindications. Pulse oximeters cannot be used.

### b) Formula for Application by the Oral Route:

The formula for injection can also be taken by the oral route or as nasal drops. The bioavailability of MB after oral administration is 72%, with peak plasma concentrations after two hours and an elimination half-life of 18 hours. MB's half-life in circulation in humans is 5 to 10 hours. The recommended dose per kg for 24 hours is identical to the dose recommended for injection. The excellent resorption of MB after oral intake makes oral intake an attractive choice.

MB can of course also be confected in dry form combined with a filler as a tablet or capsule. In view of the bitter taste, a coating of the tablet is useful to minimize gastrointestinal symptoms and to maximize patient compliance.

### c) Slow-Release Formula for Application by the Oral Route:

MB tablets, for example containing a dose of 25, 50, 100, 300 mg are confected as slow-release tablets with 2.1 gram of a mixture composed of pharmaceutical glaze, rice bran, hydroxy-propyl methylcellulose, di-calcium phosphate, stearic acid, magnesium stearate, lecithin such as soya lecithin and silica. A stomach resistant coating, confected according to recipes well known to the man of the art, protects the patient from the bitter taste and diminishes gastrointestinal irritation. The peak plasma concentration of MB is significantly prolonged due to the slow release and resorption.

1. A methylene blue (MB) compound for use in the therapeutic or prophylactic treatment, in particular in the therapeutic treatment, of hepatitis B (HBV) and/or hepatitis D (HDV) infection of a human patient.

2. The compound for use of claim 1, wherein the compound is acting by its antiviral efficacy.

3. The compound for use of claim 1 or 2, wherein the compound is applied to the patient, in particular to the infected patient, by the oral route, intranasal application, through intravenous, subcutaneous or intra muscular injection, by the rectal or nebulizer route or any combination thereof, in particular by the oral and intravenous route.

4. The compound for use of anyone of the claims claim 1 to 3, wherein the daily dose of said compound is in the range of 0.1 mg to 20 mg per kg bodyweight of the human patient.

5. The compound for use of anyone of the claims 1 to 4, wherein said compound is administered
a) orally, particularly in a daily dose of 0.1 to 20, more particularly 0.5 to 7.5, even more particularly 1 to 5, most particularly 2 to 4 mg/kg bodyweight per day of the patient; or
b) by injection particularly in a daily dose of 0.1 to 20, more particularly 0.5 to 7.5, even more particularly 1 to 5, most particularly 2 to 4 mg/kg bodyweight per day of the patient; or
c) rectally, particularly in a daily dose of 0.1 to 20, more particularly 0.5 to 7.5, even more particularly 1 to 5, most particularly 2 to 4 mg/kg bodyweight per day of the patient.

6. The compound for use of anyone of the claims 1 to 5, wherein said compound is administered by a combination of at least two routes of administration selected from the group of oral, intravenous, subcutaneous, intra muscular, intranasal or nebulizer route, particularly by a combined daily dose of 0.1 to 20, more particularly 0.5 to 7.5, even more particularly 1 to 5, most particularly 2 to 4 mg/kg bodyweight per day of the patient.

7. The compound for use of anyone of the claims 3 to 6, wherein the oral formulation is encapsulated.

8. The compound for use anyone of the claims 3 to 7, wherein the oral formulation allows for a slow release of methylene blue.

9. The compound for use of anyone of the claims 1 to 8, wherein the viral infection is caused by a Hepatitis B virus; or wherein the viral infection is caused by a simultaneous or sequential infection through a Hepatitis B virus and a Hepatitis D virus.

10. The compound for use of anyone of the claims 1 to 9, which is applied in viral infection induced distributive shock acting on vasoconstriction of small vessels due to its effect on nitric oxide.

11. The compound for use of anyone of the claims 1 to 10, being applied concomitantly, in particular simultaneously or sequentially in any order, with one or more further therapeutic agents, in particular selected from interferon alpha-2a, PEG-ylated interferon alpha-2a, lamivudine, adefovir, tenofovir disoproxil, tenofovir alafenamide, telbivudine, bulevirtide and entecavir.

12. The compound for therapeutic use according to anyone of the claims 1 to 11, wherein the treatment is performed over a period of at least one day, particularly for at least one month, more particularly for 3 to 24 months, even more particularly for 6 to 18 months, most particularly 12 months, or until a negative virological test result for the presence of HBV and/or HDV in the patient is obtained.

13. The compound for use according to anyone of the claims 1 to 12, wherein the treatment is performed in the absence of an external, in particular extra-corporal, high-energy light source activating methylene blue.

14. The compound for use according to anyone of the claims 1 to 13, wherein the compound is applied in the form of a liquid pharmaceutical composition.

15. The compound for use according to claim 14, wherein said pharmaceutical composition is in liquid form, comprising in a pharmaceutically acceptable carrier or diluent a viricidally effective amount of said compound.

16. The compound for use according to claim 14 or 15, wherein said pharmaceutical composition comprises said compound in a liquid pharmaceutically acceptable carrier in a proportion in the range of 0.1 to 2 wt.-%, particularly 0.5 to 1.5 wt.-%, and more particularly 0.8 to 1.2 wt.-%, and especially about 1 wt.-%, based on the total weight of the composition.

17. A pharmaceutical composition as defined in anyone of the claims 14 to 16.

18. A method for the therapeutic treatment of hepatitis B (HBV) and/or hepatitis D (HDV) infection of a human patient, which method comprises administering to the patient a viricidally effective amount of a methylene blue compound as further defined in anyone of the claims 1 to 16.

19. The use, compositions or method of anyone of the preceding claims, wherein MB or a pharmaceutically acceptable salt or hydrate thereof is applied.

20. The use or method of anyone of the preceding claims, wherein MB treatment is applied in combination with active or passive immunization, any type of antiviral antibody or any type of antiviral compound effective against HBV and/or HDV.
